(19) United States
(12) Patent Application Publication
(10) Pub. No.: US 2014/0363883 A1
(43) Pub. Date: Dec. 11, 2014

(54) Device for cell culture and direct imaging

(71) Applicant: BellBrook Labs, Madison, WI (US)

(72) Inventors: Steven Hayes, Madison, WI (US); Casey Lamers, Madison, WI (US); Ivar Meyvantsson, Madion, WI (US); Allyson Skoien, Belleville, WI (US); Elizabeth Vu, Verona, WI (US)

(21) Appl. No.: 14/302,098

(22) Filed: Jun. 11, 2014

Related U.S. Application Data
(60) Provisional application No. 61/833,741, filed on Jun. 11, 2013.

(51) Int. Cl. G01N 33/50

(52) U.S. Cl. CPC G01N 33/5005
USPC 435/288.7

(57) ABSTRACT

A cell culture device for the study of barrier function and differentiation are provided by this invention.

### BACKGROUND OF THE INVENTION

1. Field of the Invention

The invention disclosed herein relates generally to the field of assay development. Particularly, the invention provides a device for growing, differentiating, testing, and imaging cultured cells.

2. Description of Related Art

Epithelial barriers are perturbed in a wide range of human diseases including cystic fibrosis (CF), chronic obstructive pulmonary disease (COPD), skin cancer, polycystic kidney disease (PKD), respiratory infections, and dry eye (see e.g., Harris et al., Polycystic kidney disease. Annu Rev Med, 2009, 60: p. 321-37; Heijink et al., Cigarette smoke impairs airway epithelial barrier function and cell-cell contact recovery, Eur Respir J., 2012 February 39(2):419-28; Huet et al., Extracellular Matrix Metalloproteinase Inducer Modulates Epithelial Barrier Function through a Matrix Metalloproteinase-9-Mediated Occludin Cleavage: Implications in Dry Eye Disease, Am J Pathol., 2011 September; 179(3):1278-86; Jentsch et al., Chloride channel diseases resulting from impaired transepithelial transport or vesicular function, J Clin Invest, 2005. 115(8): p. 2039-46; Ko et al., Role of dermal-epidermal basement membrane zone in skin, cancer, and developmental disorders, Dermatol Clin., 2010. 28(1), p. 1-10). Epithelial barriers as well as non-epithelial barriers, such as the endothelial blood-brain barrier, are also critical for predicting bioavailabilty of experimental therapeutics (see Kim et al., In vitro Cellular Models for Nasal Drug Absorption Studies, Biotechnology: Pharmaceutical Aspects., 2008. Ch. 9: p. 216-234). Assays using immortalized cell lines, target overexpression, and/or other genetic engineering techniques do not often mimic in vivo conditions, which drives cell biologists in industry and academia towards more representative cell models (see Pezzulo et al., The air-liquid interface and use of primary cell cultures are important to recapitulate the transcriptional profile of in vivo airway epithelia, Am J Physiol Lung Cell Mol Physiol., 2011. 300(1): p. 25-31). Methods of monitoring barrier function, combined with the strong desire to limit animal testing, are of great interest and value to drug development and other industries, such as the cosmetics industry.

For example, methods of monitoring barrier function, in which primary cells undergo a full differentiation program on a permeable membrane with an air-liquid interface, can be used as models for understanding and therapeutically modulating a variety of diseases related to airways and skin. However, due to the complexity and laboriousness of operation and a high cost of cells and reagents, these models remain significantly underutilized in high throughput applications. Moreover, current technologies, for example Transwell® Permeable Supports devices, have limited flexibility in controlling culture conditions on the two sides of the membrane equally well and do not allow for cells to be cultured simultaneously on both sides of the membrane.

Therefore, there remains a need for methods that allow for modeling of epithelial barriers that can be adapted for a variety of cells, with or without an air-liquid interface, to be used for high throughput applications for, but not limited to, drug discovery, drug permeability studies, and basic research.

### SUMMARY OF THE INVENTION

It is against the above background that the present invention provides certain advantages and advancements over the prior art.

Although this invention disclosed herein is not limited to specific advantages or functionality, the invention provides a cell culture device, comprising one or more culture units within a culture insert, wherein the one or more culture units comprise an apical compartment and a basal compartment, wherein the device allows for growth, differentiation, and imaging of cultured cells.

In some aspects, the culture insert is removable and invertible.

In some aspects, the culture insert is between 1 and 5 mm thick.

In some aspects, the culture insert is capable of reagent exchange in the apical compartment and in the basal compartment using handheld or automated, robotic dispensing devices.

In some embodiments, the device disclosed herein further comprises (a) a tip landing pad; and (b) a volume valve.

In some aspects, the one or more culture units of the device disclosed herein comprise a porous membrane, wherein the porous membrane supports the growth of the cultured cells.

In some aspects, the porous membrane is positioned between the cultured cells and a growth medium in the basal compartment of the culture insert.

In some aspects, the porous membrane is opaque.

In some aspects, the porous membrane is translucent.

In some aspects, the porous membrane allows for visualization of the cultured cells.

In some aspects, the culture insert of the device disclosed herein is covered with a film.

In some aspects, the film comprises holes that define a culturable area and retains culture media within the culture units.

In some aspects, the film is less than 200 microns thick.

In some aspects, the culture insert of the device disclosed herein comprises regions of hydrophobicity, wherein the regions retain culture media within the culture units.

In some aspects, the imaging is performed with an inverted microscope or a high content analysis instrument.

In some aspects, the cultured cells are positioned in the basal compartment of the one or more culture units of the device disclosed herein, wherein the cultured cells are less than 200 microns from a lens on an inverted microscope.

In some embodiments, the device disclosed herein further comprises a lid, wherein the lid is optimized to minimize evaporation.

In some embodiments, the device disclosed herein further comprises: (a) a culture tray, wherein the culture tray is optimized for consistent growth conditions and; (b) an interchangeable imaging tray, wherein the imaging tray comprises a thin bottom less than 200 microns, wherein the thin bottom allows for high quality imaging.

In some aspects, the lid is a suitable culture and/or imaging tray.

In some aspects, the culture insert of the device disclosed herein comprises tabs for manual inverting, handling, and/or alignment within a culture and/or imaging tray.

In some aspects, the culture insert of the device disclosed herein comprises tabs for robotic inverting, handling, and/or alignment within a culture and/or imaging tray.

In some embodiments, the device disclosed herein is suitable for high-throughput methods.

In some embodiments, the device disclosed herein is suitable for robotic automation.

In some embodiments, the one or more culture units of the device disclosed herein are capable of the growth, differentiation, and imaging of multiple cultures within a single device.

In some embodiments, the device disclosed herein allows for the growth, differentiation, and imaging of epithelial cells.

In some embodiments, the device disclosed herein allows for the growth, differentiation, and imaging endothelial cells.

In some embodiments, the device disclosed herein allows for an assay of substances transported or secreted into the apical compartment or the basal compartment of the culture insert.

In some embodiments, the diameter of the one or more culture units of the device disclosed herein is between 0.5 and 3.0 mm.

In some embodiments, the diameter of the one or more culture units of the device disclosed herein is 1.55 mm.

In some embodiments, the one or more culture units of the device disclosed herein contain between 4,000 and 12,000 cultured cells.

In some embodiments, the one or more culture units of the device disclosed herein contain 4,000 or less cultured cells.

In some aspects, the device disclosed herein comprises 96 culture units.

In some aspects, the device disclosed herein comprises 384 culture units.

These and other features and advantages of the present invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1A shows a culture tray comprising a two-sided removable and invertible culture insert, which comprises 96 culture units, and a lid in the “off” position. FIG. 1B shows the culture tray in FIG. 1A with the lid in the “on” position.

FIG. 2A shows a cross-sectional view of a culture insert, with the apical and basal sides of the insert labeled (membrane not shown for clarity). FIG. 2B shows an apical compartment of a culture unit. FIG. 2C shows the basal side of a culture insert and tabs by which the insert can be handled. FIG. 2D shows a basal compartment of a culture unit within a culture insert (membrane not shown for clarity).

FIG. 3A indicates areas in apical compartments of a culture insert for collagen and cell loading as well as for media addition and removal. FIG. 3B indicates an area in a basal compartment of a culture insert for media addition and removal.

FIG. 4A shows a cross-sectional view of a culture unit with cells growing upon a membrane. FIG. 4B shows an apical compartment of a culture unit connected to an adjacent loading zone by a microchannel.

FIG. 5A shows a culture insert with its apical side facing upwards, which can be covered with a lid and placed in a culture tray for cell culture. FIG. 5B shows a culture insert with its basal side facing upwards, which can be covered with a lid and placed in an imaging tray for microscopy.

FIG. 6A shows a slide-sized culture device with 6 culture units. FIG. 6B shows the culture device in 6A contained within an anodized aluminum slide housing, and FIG. 6C shows the components of a slide housing assembly.

FIG. 7A is a representative image of normal human bronchial epithelial (NHBE) cells growing upon a membrane in a culture unit, which is quantified for the percentage of the membrane area occupied by ciliated cells. FIG. 7B shows cilia density in images acquired from the edge and center of commercially available well-comprising cell culture inserts, Transwell® Permeable Supports.

FIG. 8 shows representative images of cells stained for β-tubulin and DNA, which have been grown in the device of the instant application and upon a membrane of a Transwell® Permeable Supports insert.

FIGS. 9A and 9B show flat and wrinkled membranes, respectively. FIG. 9C is an image taken of a wrinkled membrane, which shows that only a subset of cells is in focus.

FIGS. 10A and 10B show the movement of fluorescent beads upon addition to pancreatic [A1] adenocarcinoma (BxPC3) cells and human bronchial epithelial (HBE) cultures, respectively. FIG. 10C shows IL-8 levels of HBE cells treated with TNF-α or with a control.

FIG. 11A shows the BxPC3 cells treated with or without transforming growth factor beta (TGF-β) that have been stained with 4′,6-diamidino-2-phenylindole (DAPI) and with a monoclonal antibody for Smad3, which translocates to the nucleus upon activation by TGF-β. FIG. 11B quantifies nuclear Smad3 of the cells imaged in FIG. 11A.

FIG. 12A shows a cross section of a Transwell® Permeable Supports insert in a well and indicates the apical compartment of the insert and the basal media reservoir. FIG. 12B shows a cross section of an instantly claimed cell culture unit within a culture insert and indicates the apical and basal compartments of the culture unit.

Skilled artisans will appreciate that elements in the figures are illustrated for simplicity and clarity and have not necessarily been drawn to scale. For example, the dimensions of some of the elements in the figures can be exaggerated relative to other elements to help improve understanding of the embodiment(s) of the present invention.

### DETAILED DESCRIPTION OF THE INVENTION

All publications, patents and patent applications cited herein are hereby expressly incorporated by reference for all purposes.

Before describing the present invention in detail, a number of terms will be defined. As used herein, the singular forms “a”, “an”, and “the” include plural referents unless the context clearly dictates otherwise. For example, reference to a “nucleic acid” means one or more nucleic acids.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the term “suitable” is used to indicate appropriateness for a particular purpose or task.

The Culture Device

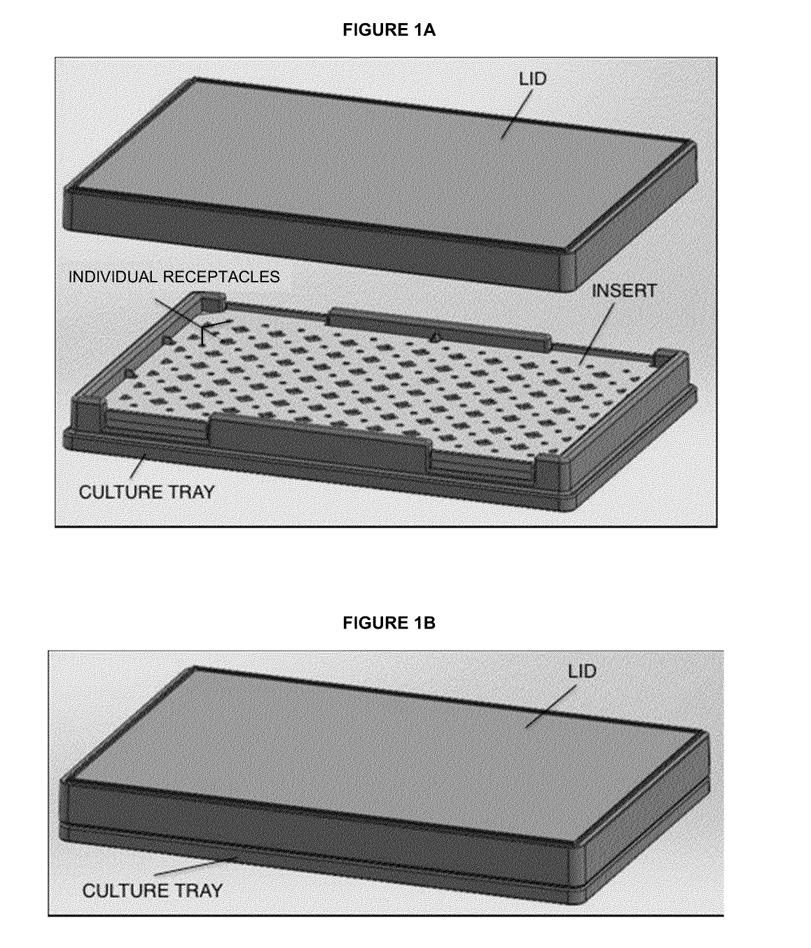

This invention disclosed herein relates to a device for growing, differentiating, and imaging cultured cells. In some embodiments, the cell culture device disclosed herein comprises a culture tray housing a two-sided culture insert and, optionally, a lid (FIG. 1). The two sides of the insert are referred to herein as the apical side (also “upper” or “top” side) and the basal side (also “lower” or “bottom” side.) The culture tray, culture insert, and lid can be biocompatible thermopolymers not limited to polycarbonate, polyethylene terphlalate, polystyrene, cyclic olefin copolymer (COP), polymethyl metacrylate (PMMA), and polypropylene. In some instances, the insert can be made of plastic with a laser sorptive material such as carbon black. The insert can be flattened by a method not limited to stacking it between metal plates, clamping the stack, and heating it in a convection oven overnight.

The culture insert (also referred to as the “insert”) can be between 1 and 5 mm thick and comprises one or more culture units for growing cells. As used herein, the terms “culture unit” and “unit” can be used to describe an individual entity for culturing cells within the insert. For example, the culture insert can comprise 1, 4, 6, 12, 48, 96, or 384 culture units. In some embodiments, the diameter of the culture unit is between 0.5 and 3.0 mm. In some embodiments, the diameter of the culture unit is 1.55 mm.

A culture unit comprises a porous membrane, which supports growth of cells under a variety of conditions. The membrane can be composed of, for example, but not limited to, polycarbonate, polyester terphalate, polystyrene, or cyclo-olefin polymers. The membrane thickness can range from 3 μm to 40 μm. In some embodiments, the membrane is between 10 μm and 20 μm thick. The pore size of the membrane can range from 0.01 μm to 10 μm. In some embodiments, the pore size is 0.45 μm. The pore density of the membrane (the number of pores per cm²) can range from 1×10⁴ to 1×10⁸ per cm². In some embodiments, the membrane pore density is from 1×10⁶ to 1×10⁷ per cm². The porosity of the membrane (the total volume of the pores as a percentage of the total volume of the membrane) can range from 1×10⁻⁷ percent to as high as 80-90%. In some embodiments, the porosity ranges from 0.2-40%.

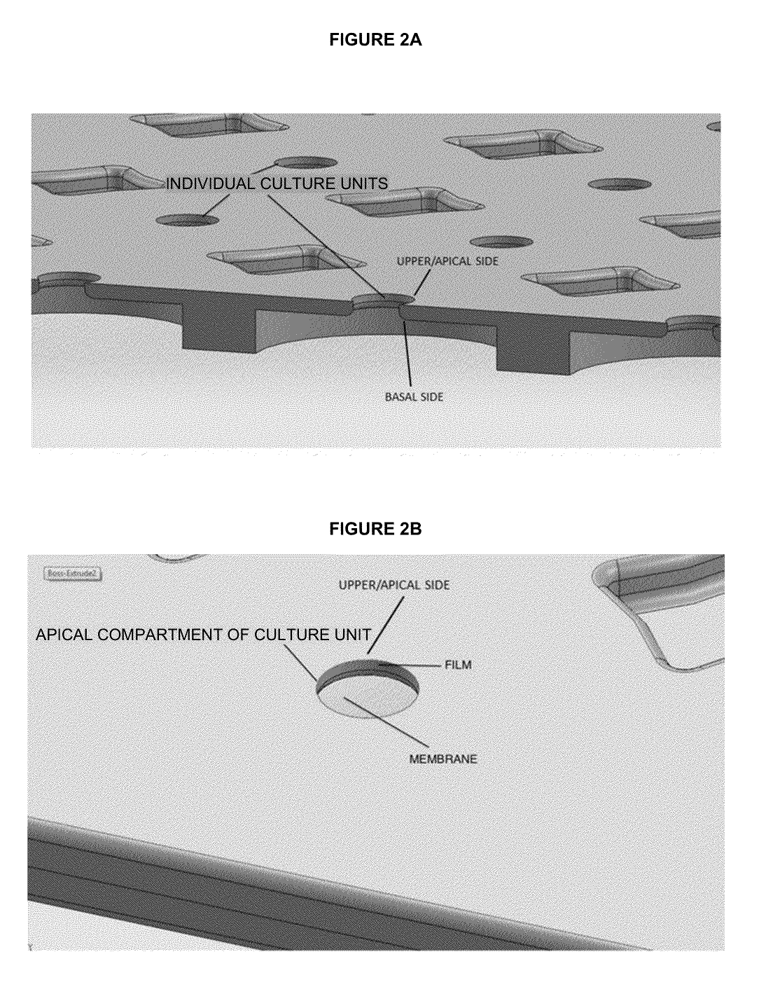

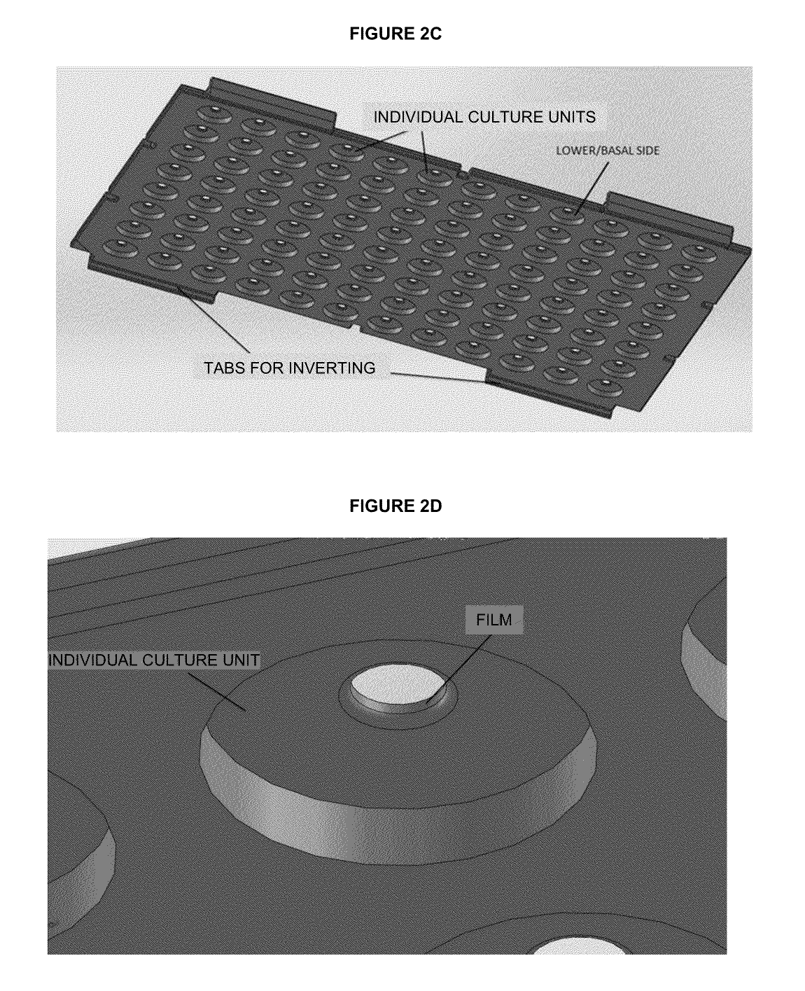

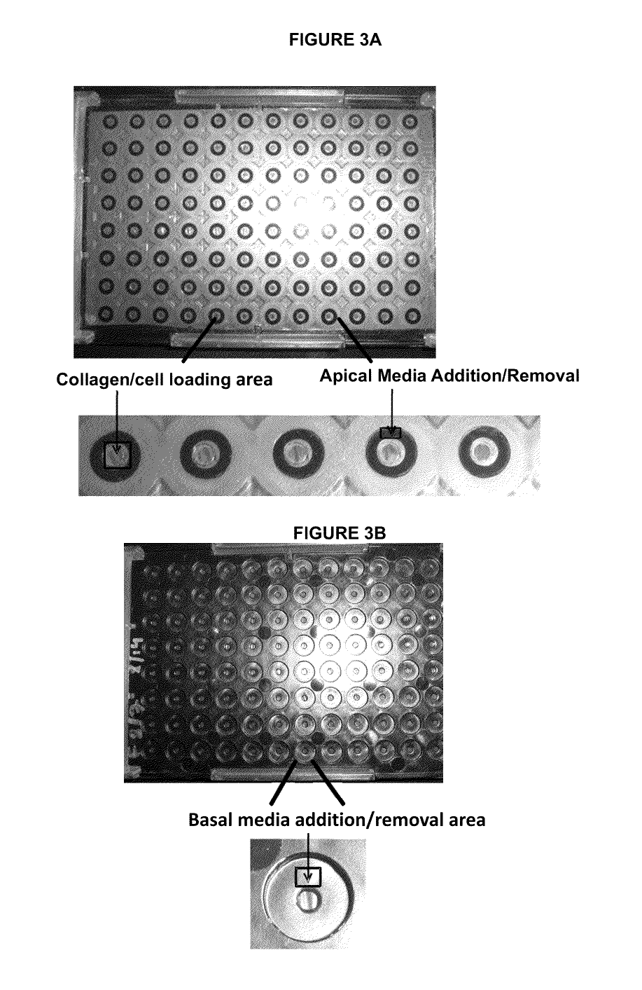

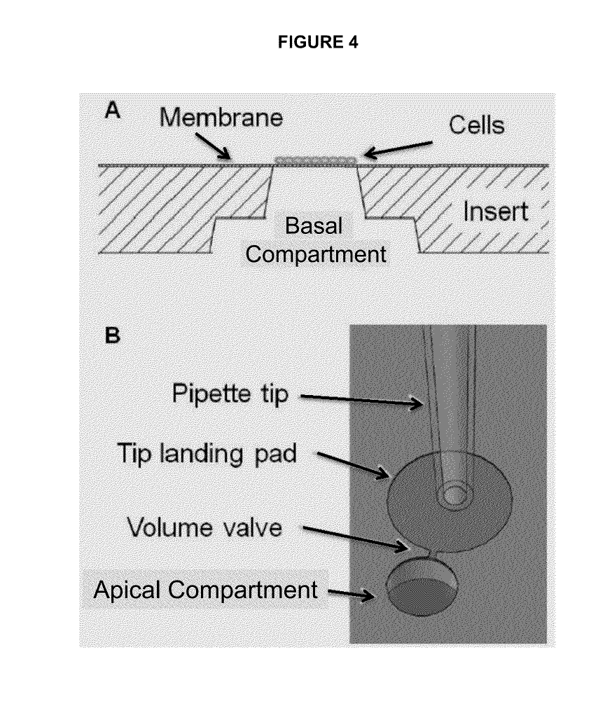

The membrane divides a unit into two discrete compartments, an apical compartment (also referred to as an “upper” or “top” compartment) and a basal compartment (also referred to as a “lower” or “bottom” compartment) (FIGS. 2, 3). As used herein, the term “compartment” refers to a self-contained space within a culture unit. The apical and basal compartments can comprise a seeding area for cells and areas for fluid loading or fluid removal (FIGS. 3, 4). In some embodiments, the volume applied to the apical compartment is 20 μL or less and the volume applied to the basal compartment is 50 μL or less. In some embodiments, the volume applied to the apical compartment is 20 μL or greater and the volume applied to the basal compartment is 50 μL or greater. In some embodiments, the membrane supports visualization of cells with an inverted microscope. In some embodiments, the membrane supports high resolution visualization of cells with an inverted microscope.

As used herein, the terms “tip landing pad,” “fluid loading zone,” and “pipetting zone” can be used interchangeably to refer to a region for pipette tip access (manual or automated), as depicted in FIG. 4. The tip landing pad can be located in the apical compartment and/or the basal compartment and allows for addition and removal of liquid from the compartments. The tip landing pad can surround (FIG. 3A) or be adjacent to (FIG. 4B) the membrane so that pipette tips and fluid do not damage, stress, or detach the cells. The tip landing pad can be used for the addition or removal of liquids not limited to media, drugs, phosphate buffered saline, antibodies, stains, and other reagents. As used herein, the term “reagent exchange” refers to the removal of one reagent or the removal of one reagent and the subsequent addition of a second reagent. If the tip landing pad is located adjacent to the membrane, a narrow, hydrophilic region or a short, hydrophobic region (referred to as a “microchannel” or “volume valve”) is placed between the tip landing pad and the membrane (FIG. 4B). The microchannel provides an energy barrier to prevent the cell-containing droplet from spreading into the tip landing pad. When a second reagent (i.e. media or an assay reagent used to detect cellular processes) is deposited onto the dry, hydrophilic fluid loading zone, the cell-containing droplet and second reagent-containing droplet coalesce. In the non-limiting embodiment, the volume and surface area of the second reagent must be substantially greater than the volume and surface area of the cell-containing droplet. Once the droplets coalesce, removal of liquid from the tip landing pad results in little or no residual liquid and minimal or no disruption of the cell monolayer. In some embodiments, the aforementioned technique is used for methods not limited to media changes, removal of waste products, and addition of assay reagents that can be inhibited by residual liquids of previous incubation steps.

As used herein, the term “culturable area” refers to the location within a culture unit wherein cells are seeded and grown. In some embodiments, a thin film for fluid containment within compartments is layered on the apical side of the insert (FIG. 2). The film can be 20-2000 μm thick. In some embodiments, the film is 100-200 μm thick. The film can comprise a standard thermoplastic not limited to polycarbonate, polyester terphalate, polystyrene, and cycloolefin polymer. The thermoplastic film comprises holes that are aligned with the culture units and are cut prior to assembly of the device using methods not limited to laser cutting and hole punching. The diameter of the holes of the film typically equals or exceeds the diameter of the culture units in the insert. The film can prevent liquids added to the tip landing pad of one compartment from dispersing into and contaminating adjacent compartments. The film can optionally comprise hydrophobic or hydrophilic regions, which control fluid path. In some embodiments, regions of the film are hydrophilic to promote wetting of the surface. In some embodiments, regions of the film are hydrophobic to prevent wetting of the surface. Control of surface wetting can be important to performance of the device. For instance, during addition of cells to the membrane, if the cell-containing droplet spreads beyond the membrane, the density of cells would be reduced. Cell density is critical for differentiation of HBE cells. Additionally, if a second reagent-containing droplet spreads beyond the pipette loading zone, the second reagent-containing droplet can coalesce with adjacent droplets.

As used herein, the term “dispensing device” refers to a tool that delivers a measured amount of liquid. In some embodiments, the dispensing devices can be handheld or automated, robotic dispensing devices. As used herein, the term “handheld robotic dispensing device” refers to, for example, a handheld Pipetman® multichannel pipettor or a 12 channel hand pipettor or Pipet-Lite® XLS+Multichannel pipettor (Ranin). As used herein, the term “automated robotic dispensing device” refers to, for example, a CyBi®-Well liquid handler or DW-CyBi® well 96/25 μL liquid handler (Cybio).

In some embodiments, the culture insert of the device disclosed herein is removable and invertible, with at least two fixed positions within a culture tray. As used herein, the terms “invert” and “inversion” refer to the action of rotating a culture insert 180° about the z-axis, the term “inverted” refers to a culture insert that has been rotated 180° about the z-axis, and the term “invertible” refers to the capability of an insert to be rotated 180° about the z-axis.

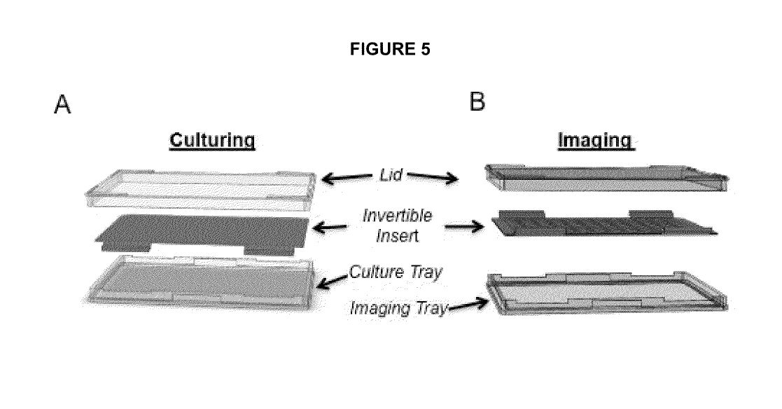

An invertible insert allows for access to both apical and basal compartments for actions such as media changes and reagent addition. For example, in some embodiments, the culture insert can be removed from the culture tray manually or by automation and inverted. In some embodiments, the lid is removed, the culture insert is inverted, and the culture insert is re-inserted into the original culture tray (FIG. 5A). The culture insert can also be inserted into a second culture tray with a thin bottom (<200 μm) that allows for high quality imaging using high resolution inverted microscopy (FIG. 5B). High quality imaging requires a thin and optically clear material such as cover glass or plastic film between the microscope objective and the sample. In the device disclosed herein, high quality imaging has been achieved by creating a tray with a thin bottom that allows for the use of high power and high-resolution microscope objectives. The tray can be further optimized to prevent evaporation, which can interfere with live-cell imaging. As such, tray optimization to prevent evaporation can be achieved by methods not limited to reducing the amount of empty space inside the enclosure and reducing the gaps between edges of the lid, tray, and culture insert.

In some embodiments, the insert can be inverted while housed in a tray and covered with a lid. The tray can subsequently be removed, and the lid becomes a tray. In doing so, the insert is not directly physically manipulated, which can lead to contamination and evaporation. The insert can optionally comprise one or more protrusion(s) to facilitate removal and/or inversion. In some embodiments, the protrusions, also termed “tabs,” are located on the periphery of the insert (FIG. 2C). For example, tabs allow for a standard plate gripper to invert the insert for culture and imaging purposes.

In some embodiments, the culture insert can be removed from the culture tray for methods not limited to cell imaging. In some embodiments, the insert can be removed from the culture tray and rotated for actions not limited to cell imaging, addition or removal of media, treatment of cells with drug compounds, treatment of cells with assay reagents, and assessment of cells with instruments such as a patch clamp.

### Cell Culture and Assays

As used herein, the term “progenitor cell” refers to a cell that is able to differentiate into a specific type of cell but does not replicate indefinitely. The culture device enables one to culture and image primary cells (cells cultured directly from human or animal with a limited replication potential in vitro) and immortalized cell lines (cells that replicate indefinitely in vitro). Non-limiting examples of compatible cells include human bronchial epithelial (HBE), pancreatic [A1] adenocarcinoma (BxPC3), non-small-cell lung cancer (Calu), human colon cancer (Caco-2), and human peripheral lung epithelial 1A (HPL1A) cells.

In particular embodiments, the culture tray functions to maintain consistent cell growth conditions, i.e. at a non-fluctuating temperature and CO₂ percentage. Consistent growth conditions are achieved by minimizing evaporative losses while maintaining sufficient gas exchange. In some embodiments, cells are grown in standard environmental conditions (i.e. 37° C., 5% CO₂). In some embodiments, cells are grown in conditions other than standard environmental conditions. A lid can function to minimize evaporation of reagents not limited to media, buffer solutions, test compound solutions, permeabilization reagents, and immunostaining reagents.

As used herein, the term “high throughput method” refers to a method that generally employs automation and is required when the number of tests of procedures to be performed exceeds the ability of manual labor to accomplish it economically. For cellular assays, including human bronchial epithelial models, only dozens or possible hundreds of assays can be currently performed in a single experiment. This is due to the limitations of human intervention, as well as the limited availability of primary cells from patients with a particular disease. However, the cell culture device disclosed herein enables throughput increases of at least 10-100 fold over current methodologies. Examples of high throughput methods in drug discovery include biochemical assays, which can enable over one million samples to be processed in a single day.

The device disclosed herein can be used to perform assays not limited to co-culture assays, differentiation assays, transport assays, migration assays, and invasion assays. For example, the device can be used for co-culture assays that differentiate the effects of soluble paracrine factors from those of direct cell-cell contact. Co-culturing assays have allowed, for example, differentiated sheets of hepatocytes to be formed from human mesenchymal stem cells using liver tissue slices to supply soluble factors (see e.g., Ong et al., Hepatic differentiation potential of commercially available human mesenchymal stem cells, Tissue Eng, 2006. 12(12): p. 3477-85; Hong et al., In vitro differentiation of human umbilical cord blood-derived mesenchymal stem cells into hepatocyte-like cells, Biochem Biophys Res Commun, 2005. 330(4): p. 1153-61; Lee et al., In vitro hepatic differentiation of human mesenchymal stem cells, Hepatology, 2004. 40(6): p. 1275-84).

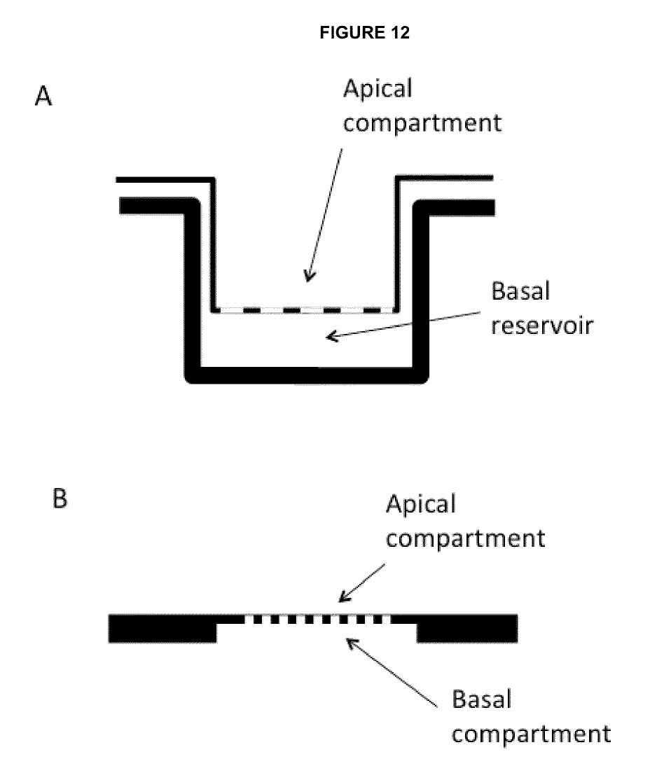

Conventional methods, such as those using well-comprising cell culture inserts (i.e. Transwell® Permeable Supports devices), cells and media are added directly to the apical (top) side of the membrane and the basal (bottom) side of the membrane is exposed to media in a well of a separate multiwell plate (FIG. 12A). As such, Transwell® Permeable Supports devices do not allow the culturing of cells on the basal side of the membrane; thus, it is not possible to study interactions between cells growing on the two sides of the membrane. The culture device described herein, however, can be used to culture a single cell type or to co-culture multiple cell types, including primary and immortilized cells. In both aspects, it is important to be able to independently control the culture conditions, including the cell type, the media, and the reagents, in the apical and basal compartments of a culture unit. With the device of the instant application, the culture insert is invertible, which facilitates the co-culturing of cells and allows for control of culture conditions in the apical and basal compartments of a culture unit. In contrast to Transwell® Permeable Supports inserts, a separate multiwell plate is not needed and no media reservoirs are created when using the device of the instant application (FIG. 12B).

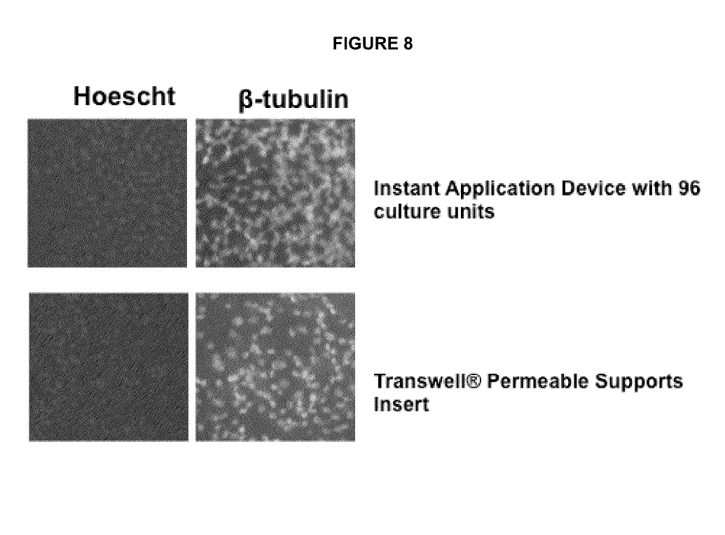

The device described herein can be used to culture cells at the air-liquid interface (ALI) and allows for a substantial reduction in the number of cells required for ALI in vitro modeling. In some embodiments, between 4,000 and 12,000 normal human bronchial epithelial (NHBE) cells can be used to create a fully differentiated airway epithelial culture using the cell culture device disclosed herein. In some embodiments, less than 4,000 NHBE cells can be used to create a fully differentiated airway epithelial culture using the cell culture device disclosed herein. In some embodiments, 10,000 NHBE cells are seeded onto the membrane of the cell culture device disclosed herein (see Example 2; FIG. 8).

As few as 4,000 primary HBE cells can be used to create a fully differentiated airway epithelial culture using this device. Conventional methods, such as those using well-comprising cell culture inserts (i.e. Transwell® Permeable Supports devices) use at least 10-fold more cells to create a differentiated airway epithelial culture (Fulcher, L. et al. Well-differentiated human airway epithelial cell cultures, Methods in Molecular Medicine, 2005. 107: p. 1830206). Lower cell numbers used in the device of the instant invention results in more effective use of primary cells from patients with airways disease. For example, a 10-fold reduction in cells seeded correlates with a ten-fold increase in the number of test compounds that can be evaluated using this device. In some embodiments, less than 4,000 immortilized cells can be seeded and grown in the device of the instant application.

The culture device disclosed herein can also be used for culturing and functional testing with intestinal epithelial cells, such as Caco-2 cells (a human epithelial colorectal adenocarcinoma cell line) that is commonly used as a model for the human intestinal mucosa to assess absorption of orally administered drugs. When cultured on a filter, Caco-2 cells differentiate to form a polarized epithelial cell monolayer that closely models the intestinal mucosa. Use of the cell culture device disclosed herein for such drug absorption assays provides similar advantages as disclosed herein with HBE cells (see Example 2 and FIG. 8), e.g., substantial reduction in the number of cells required, capability of high quality imaging, and access to the apical or basal sides of the intestinal epithelial layer.

The culture device described herein can be used for submerged cultures and ALI cultures and are capable of performing transport-related assays. For example, the device disclosed herein can be used for highly miniaturized assays for the blood-brain barrier using primary endothelial cells cultured with astrocytes and pericytes. The contents of each side of the cells can be sampled and analysed for specific components, either in the culture device or in a separate assay plate.

The culture device can be used to study infectious diseases in an airway epithelial cell model. For instance, biofilm formation in Pseudomonas aeruginosa is common for cystic fibrosis (CF) patients. The inflammatory response due to infectious disease or chronic inflammation can be studied in this device as well. Advantageously, the device disclosed herein allows for treatment of cells from both sides of the insert without dilution of secreted factors.

The culture device disclosed herein can be used to develop a skin model to advance understanding of the biology of skin as well as skin cancer. Skin models have been created using primary keratinocyte monocultures and as a co-culture system where fibroblasts are first cultured in 3D collagen (see Garlick et al., Engineering skin to study human disease-tissue models for cancer biology and wound repair, Adv Biochem Eng Biotechnol, 2007. 103: p. 207-39). Following the contraction of the collagen matrix by the fibroblasts, keratinocytes are added to the top and an ALI is introduced; under these conditions, the stratification of skin is recapitulated (see Andriani et al., Basement membrane proteins promote progression of intraepithelial neoplasia in 3-dimensional models of human stratified epithelium, Int J Cancer, 2004. 108(3): p. 348-57).

Utilization of the culture device disclosed herein further allows for measurement of information-rich phenotypic endpoints that require advanced microscopic imaging methods. Phenotypic endpoints are those that change some physical attribute of the cells and are not simply a change in the levels of a particular macromolecule. A non-limiting example of a phenotypic endpoint is a subcellular change in morphology such as the formation of stress vacuoles, autophagosomes, or loss of the perinucleolar compartment. Microscopy methods to measure phenotypic endpoints include, but are not limited to, phase contrast, differential interference contrast (DIC), epifluorescence, and more advanced imaging methods such as confocal laser scanning microscopy, spinning-disk confocal microscopy, and second-harmonic generation microscopy.

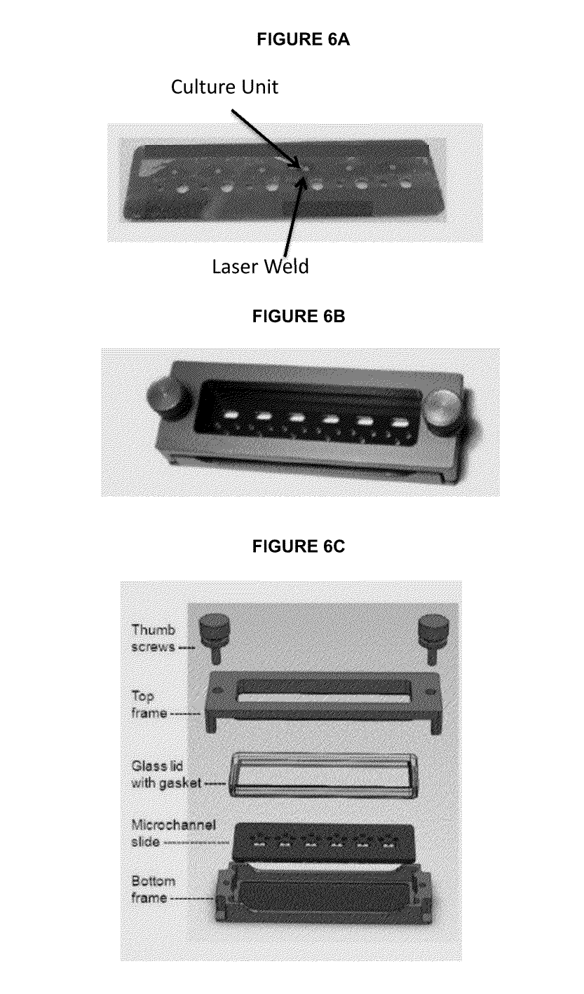

In some embodiments, a cell culture device with 96 culture units comprises the automated liquid handling compatibility (the ability to be used by robotic instruments that dispense fluids), the optical clarity (being clear to light for microscopy), and the evaporation protection (the ability to avoid loss of aqueous volume to neighboring open spaces) to enable organotypic differentiation, which is the ability of cells to differentiate into architectures resembling their cognate organ, at the air-liquid interface. In other embodiments, the cell culture device is a six-culture unit slide-sized device contained in an anodized aluminum slide housing, as shown in FIG. 6, which allows the device to fit into a standard slide insert (3 in×1 in) for a microscope stage.

In some embodiments, cells grown in the device of the instant application are analyzed by methods other than microscopy. Non-limiting examples of non-imaging readouts include airway surface liquid height, cilia beat frequency, and ion flux. The airway surface liquid height is a measure of epithelial barrier function; this height is reduced in patients with cystic fibrosis. The speed with which the cilia beat is related to mucocilicary clearance, which is critical for removing lung infections. The transportation of ions across epithelial barriers is critical for function; for instance, cystic fibrosis is due to a defect in the chlorid channel cystic fibrosis transmembrane conductance regulator (CFTR), which leads to imbalances in chloride ion transport (see Boucher et al., Na⁺transport in cystic fibrosis respiratory epithelia. Abnormal basal rate and response to adenylate cyclase activation, J Clin Invest, 1986. 78(5): p. 1245-52; Kerem et al., Identification of the cystic fibrosis gene: genetic analysis, Science, 1989. 245(4922): p. 1073-80; Knowles, et al., Abnormal ion permeation through cystic fibrosis respiratory epithelium, Science, 1983. 221(4615): p. 1067-70). Restoring CFTR function is a goal of many CF drug development efforts including the search for small molecules and effective gene therapy strategies (see Riordan et al., CFTR function and prospects for therapy, Annu Rev Biochem, 2008. 77: p. 701-26).

The invention will be further described in the following examples, which do not limit the scope of the invention described in the claims.

### EXAMPLES

The Examples that follow are illustrative of specific embodiments of the invention, and various uses thereof. They are set forth for explanatory purposes only, and are not to be taken as limiting the invention.

### Example 1

### Manufacturing of the culture device

The inserts were first injection molded from standard biocompatible thermoplastics including polycarbonate, polyethylene terphlalate, polystyrene, cyclic olefin copolymer (COP), polymethyl metacrylate (PMMA), and polypropylene. In some embodiments, the insert was made of plastic with a laser sorptive material such as carbon black, which facilitates subsequent bonding of the insert to the membrane and film. The inserts were flattened by stacking them between metal plates, clamping the stack, and heating them in a convection oven overnight.

The thermoplastic film for layering on the apical side was prepared by cutting holes for access to the membrane using laser cutting or hole punches. The membrane was cut to the size of the insert. The film and the membrane were laser welded to the insert using the approach developed by Leicester Laser (Zybko J., Laser Bonding of Plastic Microfluidic Devices). The assembly was placed into a jig to promote proper alignment of holes in the film and culture units of the insert. Pressure was placed on the assembly stack via a glass plate that also contained a chrome plated mask, which prevented areas that do not require welding from being welded during laser bonding and allowed fine micropatterning of laser bonding (Zybko J., Laser Bonding of Plastic Microfluidic Devices).

The inserts were flattened again using a convection oven as above. The surfaces of the insert, tray, and lid were treated for hydrophobic or hydrophilic patterning using a plasma generator. A removable adhesive film (mask) with holes to expose the appropriate regions of the insert was applied to the insert, and the assembly was subjected to gas plasma via an Rf generator.

The insert was assembled within the lid and tray, placed into final packaging, and the device was sterilized using standard ethylene oxide gas or gamma irradiation methods. For ethylene oxide sterilization, gas permeable packaging was used, and the sterilizations were performed with a 4 h dwell and 10.5 h cycle time. For gamma irradiation sterilization, the typical dose range for medical device processing was used.

### Example 2

### Differentiation of HNBE cells with low cell and media requirements

Normal human bronchial epithelial (NHBE) cells were seeded onto the membrane of a culture device. The conditions for seeding and culturing, selected conservatively to increase likelihood of differentiation, are listed in Table 1. Growth of cilia on the cell surface is an indication of differentiation into airway epithelium; it was measured by determining levels of β-tubulin, the primary structural component of cilia.

TABLE 1
Culture conditions for NHBE cells.

| | |
|---|---|
| # Cells per Culture Unit | 10,000 |
| Basal Media Volume | 32 μL |
| Apical Volume | 12 μL |
| Media Change Frequency | Daily |
| PBS Wash | 1 per week |
| Airlift | Day 3 |
| Evaporation Control | 2 plastic plate |
| Assay Duration | 4 weeks |
| Replicate Value | 256 culture units |
| Liquid Handling | Automated |

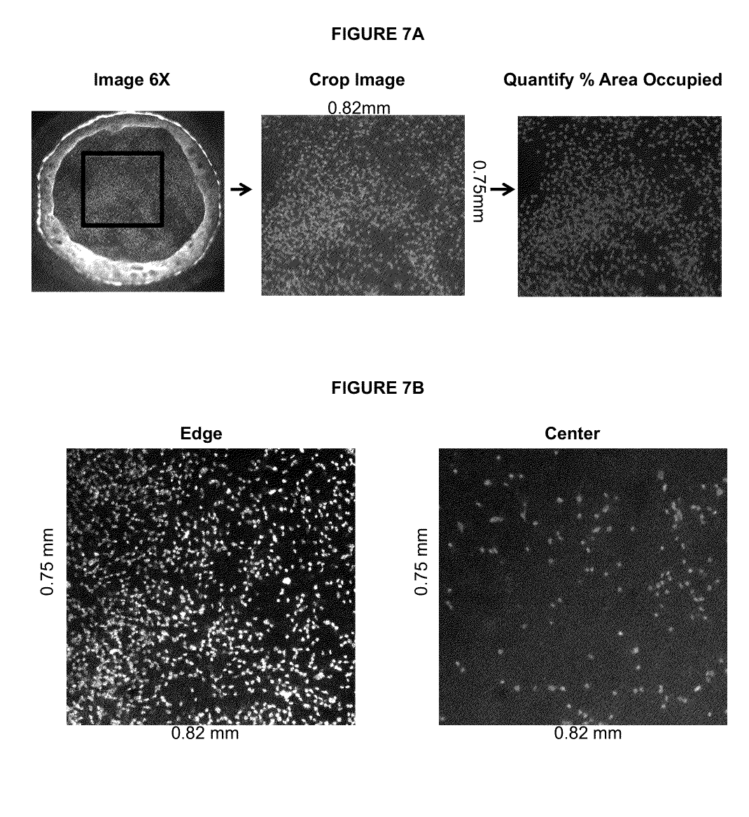

One of three plates was analyzed at 3 weeks, 6 days to assess progress of differentiation. The cells were fixed with 4% paraformaldehyde, permeabilized with Triton X-100, stained for β-tubulin using standard immunocytochemistry techniques, and analyzed for % area occupied by ciliated cells using a count nuclei algorithm (Metamorph, Molecular Devices), as depicted in FIG. 7A. Results are shown in Tables 2 and 3.

TABLE 2
Percentages of ciliated cells in air-liquid interface (ALI) device.

| | 1 | 2 | 3 | 4 | 5 | 6 | 7 | 8 |
|---|---|---|---|---|---|---|---|---|
| A | 19.7% | 20.1% | N/A | 23.8% | 27.8% | 28.0% | 29.8% | 25.9% |
| B | 22.0% | 24.1% | 23.6% | 24.4% | 29.7% | 24.2% | 25.6% | 26.7% |
| C | 25.3% | 22.6% | 17.1% | 27.1% | 26.5% | 27.1% | 26.7% | 17.8% |
| D | 26.1% | 25.9% | 28.4% | 25.8% | 23.7% | 27.4% | 26.6% | 26.5% |
| E | 20.5% | 29.0% | 27.6% | 24.6% | 28.6% | 24.0% | 24.6% | 27.0% |
| F | 21.5% | 25.8% | 21.7% | 25.9% | 29.4% | 24.4% | 23.7% | 22.0% |
| G | 27.0% | 26.3% | 21.7% | 25.5% | 26.7% | 22.7% | 26.2% | 21.4% |
| H | 10.4% | 16.3% | 22.8% | 20.9% | 24.9% | 24.4% | 24.8% | 22.8% |

TABLE 3
Analysis of ciliated cells in air-liquid interface (ALI) device.

| | Total Wells | Center Wells | Edge Wells |
|---|---|---|---|
| Average | 24% | 25% | 23% |
| Standard Deviation | 3% | 3% | 4% |
| Coefficient of Variation | 14% | 11% | 19% |
| Replicates | 63 | 42 | 21 |

At 4 weeks, the remaining two plates were with fixed with 4% paraformaldehyde, permeabilized with Triton X-100, stained for β-tubulin, and analyzed for % area occupied by ciliated cells as done above; the results of these plates are shown in Tables 4-7.

TABLE 4
Percentages of ciliated cells in plate 1.

| | 1 | 2 | 3 | 4 | 5 | 6 | 7 | 8 | 9 | 10 | 11 | 12 |
|---|---|---|---|---|---|---|---|---|---|---|---|---|
| A | 25% | 23% | 26% | 23% | 26% | 4% | 7% | 17% | 15% | 22% | 20% | 17% |
| B | 19% | 31% | 26% | 24% | 25% | 27% | 26% | 26% | 25% | 26% | 24% | 11% |
| C | 11% | 27% | 20% | 24% | 28% | 25% | 25% | 19% | 26% | 26% | 24% | 22% |
| D | 23% | 7% | 26% | 16% | 21% | 24% | 26% | 23% | 21% | 26% | 27% | 3% |
| E | 29% | 23% | 26% | 23% | 26% | 21% | 26% | 24% | 22% | 25% | 15% | 22% |
| F | 5% | 23% | 10% | 27% | 25% | 25% | 26% | 30% | 20% | 25% | 25% | 29% |
| G | 10% | 1% | 27% | 25% | 23% | 26% | 24% | 26% | 26% | 27% | 27% | 4% |
| H | 1% | 20% | 3% | 2% | 23% | 24% | 21% | 1% | 16% | 22% | 1% | 7% |

TABLE 5
Analysis of ciliated cells in plate 1.

| | Total | Center | Edge |
|---|---|---|---|
| | Culture Units | Culture Units | Culture Units |
| Average | 21% | 24% | 15% |
| Standard Deviation | 8% | 5% | 9% |
| Coefficient of | 38% | 21% | 59% |
| Variation | | | |
| Replicates | 96 | 60 | 36 |

TABLE 6
Percentages of ciliated cells in plate 2.

| | 1 | 2 | 3 | 4 | 5 | 6 | 7 | 8 | 9 | 10 | 11 | 12 |
|---|---|---|---|---|---|---|---|---|---|---|---|---|
| A | 16 | 21 | 2 | 18 | 2 | 5 | 18 | 5 | 20 | 18 | 10 | 9 |
| B | 14% | 18% | 24% | 25% | 23% | 20% | 25% | 17% | 26% | 18% | 18% | 22% |
| C | 10% | 13% | 21% | 11% | 23% | 18% | 18% | 22% | 16% | 21% | 17% | 23% |
| D | 1% | 25% | 19% | 24% | 23% | 19% | 20% | 20% | 20% | 21% | 23% | 22% |
| E | 1% | 21% | 13% | 16% | 25% | 23% | 25% | 22% | 26% | 24% | 27% | 23% |
| F | 22% | 18% | 20% | 20% | 23% | 22% | 16% | 23% | 24% | 25% | 20% | 19% |
| G | 25% | 9% | 4% | 21% | 22% | 14% | 14% | 18% | 14% | 20% | 14% | 15% |
| H | 0% | 4% | 1% | 2% | 22% | 23% | 21% | 24% | 18% | 22% | 3% | 1% |

TABLE 7
Analysis of ciliated cells in plate 2.

| | Total | Center | Edge |
|---|---|---|---|
| | Culture Units | Culture Units | Culture Units |
| Average | 17% | 20% | 13% |
| Standard Deviation | 7.1% | 4.5% | 8.7% |
| Coefficient of | 41% | 23% | 65% |
| Variation | | | |
| Replicates | 96 | 60 | 36 |

In each of the plates, there is more variability in the number of ciliated cells formed for the edge culture units than for the center culture units. To compare these data to the commercially available well-comprising cell culture insert, Transwell® Permeable Supports (Corning), normal human bronchial epithelial (NHBE) cells were seeded and cultured according to the conditions listed in Table 8, and the % area occupied by ciliated cells is shown in Table 9.

TABLE 8
Culture conditions for NHBE cells in a well-comprising
cell culture insert, Transwell ® Permeable Supports.

| | |
|---|---|
| # Cells per Well | 200,000 |
| Basal Media Volume | 1500 μL |
| Apical Volume | 500 μL |
| Media Change Frequency | Daily |
| PBS Wash | 1 per week |
| Airlift | Day 3 |
| Evaporation Control | None |
| Assay Duration | 4 weeks |
| Replicate Value | 2 wells |
| Liquid Handling | Manual |

TABLE 9
Analysis of ciliated cells in well-comprising Transwell ®
Permeable Supports cell culture inserts.

| | All Images | Center Images | Edge Images |
|---|---|---|---|
| Average | 8.4% | 3.3% | 14% |
| Standard Deviation | 5.4% | 0.5% | 0.9% |
| Coefficient of Variation | 65% | 15% | 7% |
| # of Images | 12 | 6 | 6 |

As shown in Tables 2-7 and 9, the average number of ciliated cells per culture unit area, a key marker for the extent of differentiation, is more than two-fold higher when cultured in the device of the instant application (19% average) than when cultured in the Transwell® Permeable Supports cell culture inserts (8% average). These results are statistically significant with a p-value of <0.05 using a student's t-test. FIG. 8 shows representative images of cells grown in a culture unit of the device of the instant application and in a well of a Transwell® Permeable Supports Insert, which have been stained for β-tubulin and the DNA stain, Hoechst. In these images, it is apparent that a greater percentage of ciliated cells is achieved with the device of the instant application.

The higher degree of differentiation in the culture device of the instant application is advantageous for more closely simulating the in vivo phenotype, and higher levels of differentiation increase functionality for mucociliary clearance, which is important for disease modeling. The higher degree of differentiation is surprising and unexpected since the culture device of the instant application supports differentiation of NHBE cells with substantially lower cell and media requirements as compared to, for example, Transwell® Permeable Supports cell culture inserts. These results are also surprising and unexpected because of the lower number of progenitor cells present in the group of cells seeded into the culture units of the device of the instant application. Although intra-replicate variability was higher in the device of the instant application (CV 40%, 192 culture units) compared to the Transwell® Permeable Supports cell culture inserts (CV 15% center images, 7% edge images, 2 wells), there was higher intra-well variability using Transwell® Permeable Supports cell culture inserts (CV 65% all images). Cilia density was greater in the center images than in the edge images of the Transwell® Permeable Supports cell culture inserts, as shown in FIG. 7B. As such, smaller surface area of the membranes of the instant application advantageously allows for more homogeneous cell growth than the larger surface area of the membranes of the Transwell® Permeable Supports inserts.

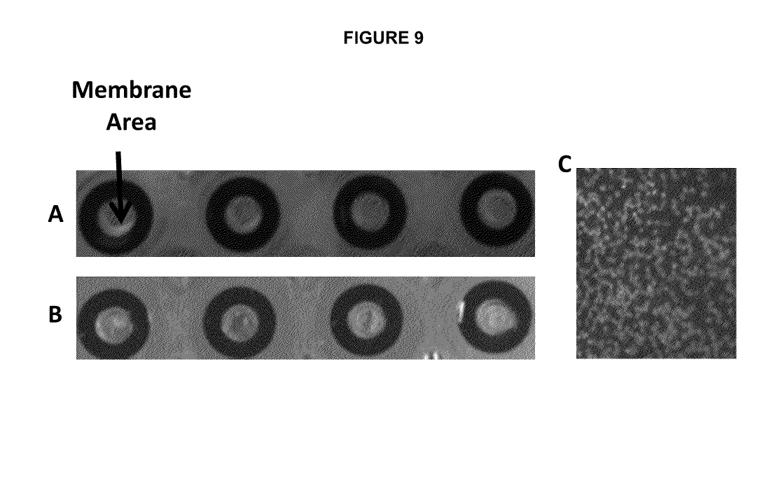

Variability of differentiation with the device of the instant application is related to insert flatness or evaporation. A flatter insert, such as the insert disclosed herein, correlates with a more uniform distribution of cells and reagents, thereby resulting in less variability. In comparison, the membrane in the current commercially available devices is wrinkled (FIG. 9B). Wrinkled membranes adversely affect high power imaging since not all of the cells on the membrane can be in the same focal plane (FIG. 9C).

### Example 3

### Monitoring of Cilia Beat and Mucocilary Clearance of Differentiated HBE Cells

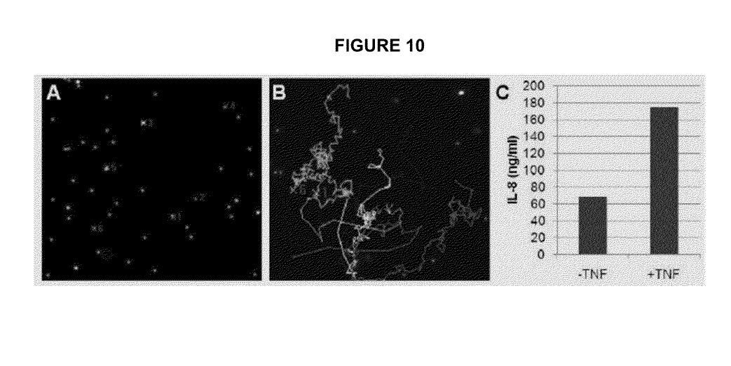

Pancreatic [A1] adenocarcinoma (BxPC3) cells and human bronchial epithelial (HBE) cells were cultured for 3 weeks in a culture device manufactured as in Example 1. The cells were assayed using a fluorescent bead-based method described by Matsui and Randell (Matsui, H., S. H. Randell, et al., Coordinated clearance of periciliary liquid and mucus from airway surfaces. 1998. J Clin invest 102(6): 1125-1131). and time-lapse images over 3 hours were taken of the cultures using a Nikon inverted TE-2000 microscope. Tracking of the beads over time revealed a clear net movement in the HBE cultures (FIG. 10B) that was not present in a culture of non-ciliated BxPC3 cells (FIG. 10A). Thus, the culture device described herein was used to monitor mucociliary clearance, the ability of the beating cilia to move particles such as bacteria suspended in the mucus, which is an important function of ciliated HBE cells.

Differentiated HBE cultures have also been reported to release pro-inflammatory cytokines in response to factors such as TNF-α, IL-1β, and lipopolysaccharide (LPS) (Pezzulo et al.). To probe this function, differentiated HBE cells were treated with 10 ng/ml TNF-α for 12 h, and the media was subsequently subjected to ELISA for IL-8 (KHC0081, Invitrogen, Dechecchi et al. Anti-inflammatory effect of miglustat in bronchial cells, 2008. J Cyst Fibros. 7(6): 555-565). In each experiment, IL-8 levels were at least two-fold higher in the TNF-α treated cells compared to untreated control, and absolute concentrations of TNF-α were similar to reported literature values (FIG. 10C) (see Pezzulo et al., The air-liquid interface and use of primary cell cultures are important to recapitulate the transcriptional profile of in vivo airway epithelia, Am J Physiol Lung Cell Mol Physiol. 300(1): p. L25-31). These results demonstrate that the HBE cultures are differentiating and functioning normally in a preferred embodiment of the invention. Furthermore, surprisingly and unexpectedly, the degree of differentiation, as measured by differentiated ciliated cells, in the device disclosed herein exceeded that of the standard larger scale device, a 12-well cell culture insert or Transwell® Permeable Supports cell culture inserts device with a 12 mm diameter. These advantageous results (i.e., the degree of differentiation of the microscale device exceeding that of macroscale devices) are surprising and unexpected because environmental conditions are harder to control on the microscale.

### Example 4

### Monitoring of Endogenous Transcription Factor Subcellular Localization

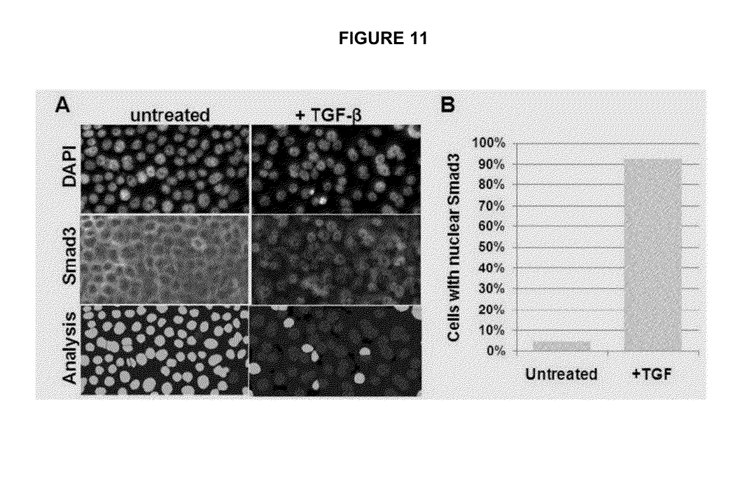

2,500 BxPC3 cells were plated onto a membrane of a culture device manufactured as described in Example 1 and grown for 24 h. The cells were then treated with or without 10 ng/mL TGF-β for 24 h and subsequently fixed with methanol and stained with a rabbit monoclonal antibody for Smad3 (C67H9, Cell Signaling Technology) and with 4′,6-diamidino-2-phenylindole DAPI (Cell Signaling Technology) using standard immunocytochemistry protocols. Smad3 is a transcription factor known to translocate from the cytoplasm to the nucleus during TGF-β signalling (see Fink et al., TGF-beta-induced nuclear localization of Smad2 and Smad3 in Smad4 null cancer cell lines, Oncogene, 2003. 22(9): p. 1317-23). The cells were imaged using a Nikon TE2000 microscope equipped with a 40× (N.A.=1.3, W.D.=0.2 mm) objective through a coverglass thin polystyrene film (FIG. 7A). Cells were analyzed for nuclear translocation using automated multiwavelength cell scoring (Metamorph, Molecular Devices), as shown in the lower panels of FIG. 11A. Quantitation of automated image analysis shows dramatically increased Smad3 nuclear translocation in response to TGF-β (FIG. 11B). These results demonstrate that the image quality in the device disclosed herein is sufficient to support high-content subcellular localization assays for endogenous transcription factors.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

What is claimed is:

1. A cell culture device, comprising one or more culture units within a culture insert, wherein the one or more culture units comprise an apical compartment and a basal compartment, wherein the device allows for growth, differentiation, and imaging of cultured cells.

2. The device of claim 1, wherein the culture insert is removable and invertible.

3. The device of claim 1, wherein the culture insert is between 1 and 5 mm thick.

4. The device of claim 1, wherein the culture insert is capable of reagent exchange in the apical compartment and in the basal compartment using handheld or automated, robotic dispensing devices.

5. The device of claim 1, wherein the one or more culture units comprise a porous membrane, wherein the porous membrane supports the growth of the cultured cells.

6. The device of claim 5, wherein the porous membrane is positioned between the cultured cells and a growth medium in the basal compartment of the culture insert.

7. The device of claim 5, wherein the porous membrane is opaque and/or translucent, wherein the porous membrane allows for visualization of the cultured cells.

8. The device of claim 1, wherein the culture insert is covered with a film, wherein the film comprises holes that define a culturable area and retains culture media within the culture units.

9. The device of claim 8, wherein the film is less than 200 microns thick.

10. The device of claim 1, wherein the culture insert comprises regions of hydrophobicity, wherein the regions retain culture media within the one or more culture units.

11. The device of claim 1, wherein the imaging is performed with an inverted microscope or a high content analysis instrument, wherein the cultured cells are positioned in the basal compartment of the one or more culture units, wherein the cultured cells are less than 200 microns from a lens on the inverted microscope or the high content analysis instrument.

12. The device of claim 1, wherein the device further comprises one or more of the following:
(a) a culture tray, wherein the culture tray is optimized for consistent growth conditions;
(b) an interchangeable imaging tray, wherein the imaging tray comprises a thin bottom less than 200 microns, wherein the thin bottom allows for high quality imaging;
(c) a lid, wherein the lid is optimized to minimize evaporation;
(d) a tip landing pad; and
(e) a volume valve.

13. The device of claim 12, wherein the lid is a suitable culture and/or imaging tray.

14. The device of claim 1, wherein the culture insert comprises tabs for manual inverting, handling, and/or alignment within a culture and/or imaging tray.

15. The device of claim 1, wherein the culture insert comprises tabs for robotic inverting, handling, and/or alignment within a culture and/or imaging tray.

16. The device of claim 1, wherein the device is suitable for high-throughput methods and/or robotic automation.

17. The device of claim 1, wherein the one or more culture units are capable of the growth, differentiation, and imaging of multiple cultures within a single device.

18. The device of claim 1, wherein the cultured cells are epithelial cells or endothelial cells.

19. The device of claim 1, which allows for an assay of substances transported or secreted into the apical compartment or the basal compartment of the one or more culture units.

20. The device of claim 1, wherein a diameter of the one or more culture units is between 0.5 and 3.0 mm.

21. The device of claim 1, wherein the one or more culture units contain between 4,000 and 12,000 cultured cells.

22. The device of claim 1, wherein the device comprises 96 culture units or 384 culture units.
